SEQUENCE LISTING (22 sequences)

SEQ ID NO: 1 - protein, 168 aa, Homo sapiens
  MISC_FEATURE (1)..(168): N-terminal domain of human osteopontin protein
  MISC_FEATURE (1)..(17): Signal sequence of human osteopontin protein
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLR

SEQ ID NO: 2 - DNA, 1475 bp, Mus musculus
  misc_feature: Full length murine osteopontin nucleic acid sequence
agccaccagcagctcacactgaagaagcatccttgcttgggtttgcagtcttctgcggcaggcattctcggaggaaaccagccaaggtaagcctgcagtggcccatgaggctgcagttctcctggctgaattctgagggactaactacgaccatgagattggcagtgatttgcttttgcctgtttggcattgcctcctccctcccggtgaaagtgactgattctggcagctcagaggagaagaagctttacagcctgcacccagatcctatagccacatggctggtgcctgacccatctcagaagcagaatctccttgcgccacagaatgctgtgtcctctgaagaaaaggatgactttaagcaagaaactcttccaagcaattccaatgaaagccatgaccacatggacgacgatgatgacgatgatgatgacgatggagaccatgcagagagcgaggattctgtggactcggatgaatctgacgaatctcaccattcggatgagtctgatgagaccgtcactgctagtacacaagcagacactttcactccaatcgtccctacagtcgatgtccccaacggccgaggtgatagcttggcttatggactgaggtcaaagtctaggagtttccaggtttctgatgaacagtatcctgatgccacagatgaggacctcacctctcacatgaagagcggtgagtctaaggagtccctcgatgtcatccctgttgcccagcttctgagcatgccctctgatcaggacaacaacggaaagggcagccatgagtcaagtcagctggatgaaccaagtctggaaacacacagacttgagcattccaaagagagccaggagagtgccgatcagtcggatgtgatcgatagtcaagcaagttccaaagccagcctggaacatcagagccacaagtttcacagccacaaggacaagctagtcctagaccctaagagtaaggaagatgataggtatctgaaattccgaatttctcatgaattagagagttcatcttctgaggtcaactaaagaagaggcaaaaacacagttccttactttgcatttagtaaaaacaagaaaaagtgttagtgagggttaagcaggaatactaactgctcatttctcagttcagtggatatatgtatgtagagaaagagaggtaatattttgggctcttagcttagtctgttgtttcatgcaaacaccgttgtaaccaaaagcttctgcactttgcttctgttcttcctgtacaagaaatgcaaacggccactgcattttaatgattgttattcttttatgaataaaatgtatgtagaaacaagcaaatttactgaaacaagcagaattaaaagagaaactgtaacagtctatatcactatacccttttagttttataattagcatatattttgttgtgattattttttttgttggtgtgaataaatcttgtaacgaatgtaagga

SEQ ID NO: 3 - protein, 311 aa, Mus musculus
  MISC_FEATURE: Full length murine osteopontin protein
MRLQFSWLNSEGLTTTMRLAVICFCLFGIASSLPVKVTDSGSSEEKKLYSLHPDPIATWLVPDPSQKQNLLAPQNAVSSEEKDDFKQETLPSNSNESHDHMDDDDDDDDDDGDHAESEDSVDSDESDESHHSDESDETVTASTQADTFTPIVPTVDVPNGRGDSLAYGLRSKSRSFQVSDEQYPDATDEDLTSHMKSGESKESLDVIPVAQLLSMPSDQDNNGKGSHESSQLDEPSLETHRLEHSKESQESADQSDVIDSQASSKASLEHQSHKFHSHKDKLVLDPKSKEDDRYLKFRISHELESSSSEVN

SEQ ID NO: 4 - DNA, 1561 bp, Homo sapiens
  misc_feature: Full length human osteopontin nucleic acid sequence
agcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagaatgctgtgtcctctgaagaaaccaatgactttaaacaagagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgccta

SEQ ID NO: 5 - protein, 314 aa, Homo sapiens
  MISC_FEATURE: Full length human osteopontin protein
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 6 - protein, 267 aa, SARS-CoV-2
  MISC_FEATURE (1)..(260): Receptor binding domain of spike glycoprotein of SARS-CoV-2
  MISC_FEATURE (261)..(267): Histidine tag
LLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNGSGHHHHHHH

SEQ ID NO: 7 - protein, 435 aa, Artificial Sequence
  Human OPN-COV fusion protein
  MISC_FEATURE (1)..(167): N-terminal domain of human osteopontin protein
  MISC_FEATURE (1)..(17): Signal sequence of human osteopontin protein
  MISC_FEATURE (168)..(428): Receptor binding domain of spike glycoprotein of SARS-CoV-2
  MISC_FEATURE (429)..(435): Histidine tag
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNGSGHHHHHHH

SEQ ID NO: 8 - DNA, 1317 bp, Artificial Sequence
  OPN-COV fusion protein
atgcggatcgctgtgatttgtttctgccttctggggataacctgcgctatacccgttaaacaggcggactcaggatcaagtgaggaaaagcagctctacaacaaatatccagacgctgtcgctacttggctcaacccagaccctagccagaaacagaacctgctggctcctcaaaacgcagtttcctcagaagagactaacgatttcaaacaagagactctcccctcaaagtctaatgaatcccacgaccacatggacgacatggacgatgaggacgacgatgatcacgtagatagccaagactctatcgactcaaacgatagcgatgacgtggatgatacagacgactcacatcagtccgatgagtcacatcattctgacgaatctgacgaattggttacggactttcctacagatctcccagccacagaagttttcaccccggttgttcctaccgtcgatacgtatgatgggcggggcgactcagtcgtatatgggctgcgactgctcaaatataatgagaatgggaccatcaccgacgctgtggactgtgcattggacccgctgtccgagactaaatgtacccttaaatcttttacggtggagaaaggaatatatcagaccagtaactttcgagtccagccaacggagtctatcgtgaggttcccgaatatcaccaatctctgtccctttggggaggtgtttaacgctacgagatttgccagcgtgtatgcttggaaccggaaaaggattagcaactgtgttgcggactacagcgttctgtacaactcagcttctttttccaccttcaaatgttatggagtaagccccacaaagcttaatgacctctgttttacgaacgtgtacgccgattcctttgtcataaggggtgacgaggtaaggcaaattgcaccaggtcaaaccggcaagatagcggattacaattataaattgcccgacgactttacgggctgtgtcatagcctggaattccaataacctcgatagtaaagtaggcggtaactacaactatctctatcggttgtttcgcaaatccaacttgaaaccattcgagcgagacatatccactgagatctatcaagctgggagtaccccgtgtaacggcgtggaaggttttaattgttactttccgctgcagtcatatgggtttcaaccaacgaatggcgtgggatatcaaccatacagagtagttgttttgtcattcgaactccttcacgctcctgctactgtttgtggacccaaaaagtcaactaatggaagcgggcaccaccatcaccaccaccattgataactcgag

SEQ ID NO: 9 - protein, 170 aa, Artificial Sequence
  N-terminal domain of murine osteopontin protein
MRLQFSWLNSEGLTTTMRLAVICFCLFGIASSLPVKVTDSGSSEEKKLYSLHPDPIATWLVPDPSQKQNLLAPQNAVSSEEKDDFKQETLPSNSNESHDHMDDDDDDDDDDGDHAESEDSVDSDESDESHHSDESDETVTASTQADTFTPIVPTVDVPNGRGDSLAYGLR

SEQ ID NO: 10 - protein, 116 aa, Artificial Sequence
  N-terminal domain of murine osteopontin protein, truncated
  MISC_FEATURE: Missing amino acids 33-86 of SEQ ID NO. 9
MRLQFSWLNSEGLTTTMRLAVICFCLFGIASSQETLPSNSNESHDHMDDDDDDDDDDGDHAESEDSVDSDESDESHHSDESDETVTASTQADTFTPIVPTVDVPNGRGDSLAYGLR

SEQ ID NO: 11 - protein, 143 aa, Artificial Sequence
  N-terminal domain of murine osteopontin, without exon 4
MRLQFSWLNSEGLTTTMRLAVICFCLFGIASSLPVKVTDSGSSEEKQNAVSSEEKDDFKQETLPSNSNESHDHMDDDDDDDDDDGDHAESEDSVDSDESDESHHSDESDETVTASTQADTFTPIVPTVDVPNGRGDSLAYGLR

SEQ ID NO: 12 - protein, 159 aa, Artificial Sequence
  N-terminal domain of murine osteopontin protein, without RGD
MRLQFSWLNSEGLTTTMRLAVICFCLFGIASSLPVKVTDSGSSEEKKLYSLHPDPIATWLVPDPSQKQNLLAPQNAVSSEEKDDFKQETLPSNSNESHDHMDDDDDDDDDDGDHAESEDSVDSDESDESHHSDESDETVTASTQADTFTPIVPTVDVPN

SEQ ID NO: 13 - protein, 179 aa, Artificial Sequence
  C-terminal domain of murine osteopontin
MRLQFSWLNSEGLTTTMRLAVICFCLFGIASSLAYGLRSKSRSFQVSDEQYPDATDEDLTSHMKSGESKESLDVIPVAQLLSMPSDQDNNGKGSHESSQLDEPSLETHRLEHSKESQESADQSDVIDSQASSKASLEHQSHKFHSHKDKLVLDPKSKEDDRYLKFRISHELESSSSEVN

SEQ ID NO: 14 - protein, 7 aa, Mus musculus
  MISC_FEATURE (1)..(7): Amino acids 87-93 of murine osteopontin protein.
  MISC_FEATURE (1)..(7): Amino acids 87-93 of murine osteopontin protein
QETLPSN

SEQ ID NO: 15 - protein, 5 aa, Mus musculus
  MISC_FEATURE (1)..(5): Amino acids 160-164 of murine osteopontin protein
GRGDS

SEQ ID NO: 16 - protein, 11 aa, Mus musculus
  MISC_FEATURE: Amino acids 160-169 of murine osteopontin protein
GRGDSLAYGLR

SEQ ID NO: 17 - protein, 1273 aa, SARS-CoV-2
  MISC_FEATURE: Severe Acute Respiratory Syndrome coronavirus 2 isolate SARS-CoV-2 surface glycoprotein S
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - protein, 17 aa, Homo sapiens
  MISC_FEATURE: Signal sequence of human osteopontin protein
MRIAVICFCLLGITCAI

SEQ ID NO: 19 - DNA, 507 bp, Artificial Sequence
  Nucleic acid sequence encoding human OPN-NT plus stop codon
atgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagaatgctgtgtcctctgaagaaaccaatgactttaaacaagagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtaa

SEQ ID NO: 20 - protein, 154 aa, Artificial Sequence
  Human OPN-NT lacking exon 5
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLR

SEQ ID NO: 21 - protein, 141 aa, Artificial Sequence
  Human OPN-NT lacking exon 4
MRIAVICFCLLGITCAIPVKQADSGSSEEKQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLR

SEQ ID NO: 22 - protein, 127 aa, Artificial Sequence
  Human OPN-NT lacking exon 4 and exon 5
MRIAVICFCLLGITCAIPVKQADSGSSEEKQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLR

(19) United States
(12) Patent Application Publication
(10) Pub. No.: US 2024/0207387 A1
(43) Pub. Date: Jun. 27, 2024

(54) A VACCINE ADJUVANT FOR INFECTIOUS DISEASES

(71) Applicant: University of Cincinnati, Cincinnati, OH (US)

(72) Inventor: Georg F. Weber, Milford, OH (US)

(73) Assignee: University of Cincinnati, Cincinnati, OH (US)

(21) Appl. No.: 18/555,940

(22) PCT Filed: Apr. 20, 2022
(86) PCT No.: PCT/US2022/025516

§ 371 Date: Oct. 18, 2023

Related U.S. Application Data
(60) Provisional application No. 63/177,133, filed on Apr. 20, 2021.

(51) Int. Cl. A61K 39/145; A61K 39/00; A61K 39/215; A61K 39/39; C12N 15/86

(52) U.S. Cl. CPC A61K 39/145; A61K 39/215; A61K 39/39; C12N 15/86; A61K 2039/5252; A61K 2039/55516; A61K 2039/6031; C12N 2760/16134; C12N 2770/20034

(57) ABSTRACT

Provided herein is a vaccine adjuvant containing an N-terminal domain of osteopontin or a fragment thereof. Also provided are conjugates and fusion proteins containing the N-terminal domain of osteopontin conjugated to a pathogen or a protein derived therefrom. A method for potentiating an immune response to an immunizing antigen is also provided, the method including administering to a subject an effective amount of a vaccine adjuvant containing an N-terminal domain of osteopontin. Also provided is a method of vaccinating a subject against SARS-CoV-2, the method including administering to a subject a fusion protein containing the N-terminal domain of osteopontin and the receptor binding domain of SARS-CoV-2 spike glycoprotein. Cellular vaccines and methods of vaccinating a subject with a cellular vaccine are also provided herein.

### CROSS REFERENCE TO RELATED APPLICATION

This application claims priority to U.S. Provisional Application Ser. No. 63/177,133, filed Apr. 20, 2021, the entire contents of which are incorporated herein by reference.

### TECHNICAL FIELD

The present disclosure relates to the field of vaccinology. More specifically, the present disclosure relates to a vaccine adjuvant comprising the N-terminal domain of osteopontin (OPN-NT) and its methods of use.

### SEQUENCE LISTING

A computer-readable form (CRF) sequence listing having file name Sequence_Listing_CINO356WO.txt, created on Apr. 14, 2022, is incorporated herein by reference. The nucleic and amino acid sequences listed in the accompanying sequence listing are shown using standard abbreviations as defined in 37 C.F.R. 1.822, wherein:
SEQ ID NO: 1 corresponds to an amino acid sequence encoding human OPN-NT;
SEQ ID NO: 2 corresponds to a nucleic acid sequence encoding murine OPN;
SEQ ID NO: 3 corresponds to an amino acid sequence encoding murine OPN;
SEQ ID NO: 4 corresponds to a nucleic acid sequence encoding human OPN;
SEQ ID NO: 5 corresponds to an amino acid sequence human OPN;
SEQ ID NO: 6 corresponds to an amino acid sequence encoding the receptor binding domain of SARS-CoV-2 spike protein;
SEQ ID NO: 7 corresponds to an amino acid sequence encoding a human OPN-COV fusion protein;
SEQ ID NO: 8 corresponds to a nucleic acid sequence encoding a human OPN-COV fusion protein;
SEQ ID NO: 9 corresponds to an amino acid sequence encoding a murine OPN-NT sequence;
SEQ ID NO: 10 corresponds to an amino acid sequence encoding a truncated murine OPN-NT sequence;
SEQ ID NO: 11 corresponds to an amino acid sequence encoding a truncated murine OPN-NT sequence, wherein exon 4 is deleted;
SEQ ID NO: 12 corresponds to an amino acid sequence encoding a murine OPN-NT sequence, wherein the RGD domain is deleted;
SEQ ID NO: 13 corresponds to an amino acid sequence encoding murine OPN-CT;
SEQ ID NO: 14 corresponds to selected amino acids of murine OPN;
SEQ ID NO: 15 corresponds to selected amino acids of murine OPN;
SEQ ID NO: 16 corresponds to selected amino acids of murine OPN;
SEQ ID NO: 17 corresponds to an amino acid sequence encoding SARS-CoV-2 spike protein;
SEQ ID NO: 18 corresponds to an amino acid sequence encoding a secretion signal sequence of human OPN;
SEQ ID NO: 19 corresponds to a nucleic acid sequence encoding human OPN-NT;
SEQ ID NO: 20 corresponds to an amino acid sequence encoding human OPN-NT, wherein exon 5 is deleted;
SEQ ID NO: 21 corresponds to an amino acid sequence encoding human OPN-NT, wherein exon 4 is deleted; and
SEQ ID NO: 22 corresponds to an amino acid sequence encoding human OPN-NT, wherein exon 4 and exon 5 are deleted.

### BACKGROUND

Vaccination is the most efficient public health measure to address viral threats, including influenza and coronaviruses such as severe acute respiratory syndrome coronavirus 2 (SARS-CoV-2). However, vaccination efficiency is variable, depending on pathogen factors such as viral strain, as well as host factors such as potential impaired immune responsiveness in the elderly and the very young. In the host, the cytokine profile accompanying an immunotherapeutic regimen is an important early determinant for outcome. While traditional vaccines aim to generate a high antibody titer, both arms of the adaptive immune response—type I (cellular or cell-mediated) and type II (humoral or antibody-mediated)—contribute to an efficient antiviral reaction.

Cytokines are hormonal messengers that mediate biological processes of the immune system, including cell-mediated immunity. T helper (Th) lymphocytes expressing CD4 are prolific producers of cytokines. Th1 cells express Th1-type cytokines, such as interleukins (IL)-2 and 12 and interferon gamma (IFNγ), which are pro-inflammatory and primarily responsible for killing intracellular parasites and mediating autoimmune responses. Th2 cells express Th2-type cytokines, such as interleukins (IL)-4, 5, 10, and 13. A robust immune response should include a well-balanced combination humoral and cellular immune components, including both Th1 and Th2 responses.

A need exists for improved vaccines, adjuvants, and methods that effectively elicit both antibody- and cell-mediated adaptive immunity, with a favorable balance of Th1 and Th2 cytokines.

### SUMMARY

The cytokine osteopontin (OPN) is a proximal regulator of type I and type II adaptive immunity and may be utilized to direct the phenotype of an immune response. OPN comprises two distinct immunoregulatory domains, the N-terminal (NT) and C-terminal (CT) domains, each of which independently modulates IL-10 and IL-12 secretion from macrophages. Specifically, OPN-NT induces IL-12, which elicits a Th1 immune response, as well as IL-10, which elicits a Th2 immune response. Thus, OPN-NT induces a balance of Th1 and Th2 cytokines favorable for a robust immune response to an immunizing antigen. Accordingly, provided herein is a vaccine adjuvant and vaccine conjugates comprising OPN-NT, which potentiate an immune response to an immunizing antigen by inducing a balanced Th1/Th2 immune response in a recipient subject.

In one embodiment, a vaccine adjuvant is provided, comprising an N-terminal domain of osteopontin (OPN-NT) or a fragment thereof.

In another embodiment, a fusion protein is provided, comprising OPN-NT or a fragment thereof, conjugated to an immunogenic protein or fragment thereof derived from a pathogenic virus.

In another embodiment, a method for potentiating an immune response to an immunizing antigen in a subject is provided, the method comprising administering to the subject an effective amount of a vaccine adjuvant comprising OPN-NT or a fragment thereof.

In another embodiment, a method of vaccinating a subject against SARS-CoV-2 is provided, the method comprising administering to the subject an effective amount of a fusion protein comprising OPN-NT or a fragment thereof and a receptor binding domain of SARS-CoV-2 spike glycoprotein.

In another embodiment, a cell engineered to express a vaccine adjuvant comprising OPN-NT or a fragment thereof is provided.

In another embodiment, a vaccine is provided, the vaccine comprising a cell engineered to express a vaccine adjuvant comprising OPN-NT or a fragment thereof and a pharmaceutically acceptable carrier.

In another embodiment, a method of vaccinating a subject in need thereof is provided, the method comprising: obtaining autologous cells from the subject; transducing the autologous cells with a nucleic acid encoding OPN-NT; and reintroducing the autologous cells into the subject.

These and other objects, features, embodiments, and advantages will become apparent to those of ordinary skill in the art from a reading of the following detailed description and the appended claims.

### BRIEF DESCRIPTION OF THE DRAWINGS

The details of embodiments of the presently-disclosed subject matter are set forth in this document. Modifications to embodiments described in this document, and other embodiments, will be evident to those of ordinary skill in the art after a study of the information provided herein.

FIG. 1 depicts the immunomodulatory effects of the N-terminal domain of osteopontin (OPN-NT) and the C-terminal domain of osteopontin (OPN-CT) on type I and type II adaptive immunity.

FIG. 2 depicts a map of mouse osteopontin protein domains and describes the functions of the respective N- and C-terminal domains.

FIG. 3 depicts the composition of various osteopontin constructs, including OPN, OPN-NT, and OPN-CT constructs.

FIG. 4 is an image of an SDS-PAGE gel showing constructs 1-5 of signal-containing OPN subcloned into pcDNA3.1(+).

FIG. 5 is an image of an SDS-PAGE gel showing purified OPN and the flow-through (FT) from the nickel column used for purification. A clear band for the OPN construct is observed in the OPN lane.

FIG. 6 is an image of an SDS-PAGE gel showing signal-containing OPN-COV (A lanes) and OPN-COV without a signal sequence (B lanes) produced in recombinant adenovirus expression systems transfected into HEK293 cells.

FIG. 7 is an image of an SDS-PAGE gel showing signal-containing OPN-COV with signal sequence (7 left-hand lanes) and OPN-COV without signal sequence (7 right-hand lanes) retrieved with KpnI+XhoI from pShuttle constructs subcloned into pcDNA3.1(+) and produced via transient transfection of HEK293 cells.

FIG. 8 is an image of an SDS-PAGE gel showing total protein (T), medium supernatant (S), elution nickel column (E) fractions for medium supernatant from signal-containing adenovirus infected HEK293 cells (Ad-Sig-OPNCOV), medium supernatant from transient transfection with pcDNA3.1 construct with signal-containing OPNCOV (3.1Sig-OPNCOV), and combined cell pellets from Ad-OPNCOV-infected cells and transiently infected cells by pcDNA3.1-OPNCOV, both without signal sequence (Ad-OPNCOV+3.1-OPNCOV).

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an” and “the” include plural references unless the content clearly dictates otherwise.

As used herein, the terms “administer” or “administration” may comprise administration routes such as parenteral (e.g., subcutaneously, intradermally, intramuscularly, or intravenously), oral, intranasal, etc., so long as the route of administration results in the generation of an immune response in the subject. In specific embodiments, the administration route is an intramuscular injection.

As used herein, the term “subject” generally refers to a living being (e.g., animal or human) that is able to mount an immune response as described herein, preferably leading to the production of antibodies and/or lymphocytes that specifically bind to the immunizing antigen and/or conjugates comprising an immunizing antigen described herein. In some embodiments, a subject described herein may be a patient to be treated therapeutically (e.g., via vaccination) or may be employed as a means for generating tools (e.g., antibodies) for research, diagnostic, and/or therapeutic purposes. In a specific embodiment, the subject is a human subject.

The cytokine osteopontin (OPN) regulates type I and type II adaptive immunity at a more proximal level than most other cytokines. As such, osteopontin may be utilized to direct the phenotype of an immune response. OPN comprises two distinct immunoregulatory domains, each of which independently modulates IL-10 and IL-12 secretion from macrophages. The N-terminal domain of osteopontin (OPN-NT) induces IL-12, a cytokine that induces a Th1 immune response, as well as IL-10, a cytokine that induces a Th2 immune response. The C-terminal domain (OPN-CT), on the other hand, selectively suppresses IL-10 and the full-length osteopontin both increases IL-12 and suppresses IL-10. Given this immune profile, OPN-NT is better positioned to serve as a vaccine adjuvant than OPN-CT, which selectively suppresses IL-10, or the full-length OPN, which increases IL-12, but also suppresses IL-10.

It has become increasingly clear that the outcome of vaccination in general is decisively determined by the type of immune response induced. Immunization with inactivated virus generates a Th2 response and does not lead to heterosubtypic immunity. This can be remedied by the generation of a Th1 priming environment. Yet, a cytokine-based modulation of existing vaccination strategies that efficiently directs the immune system to induce a combined cellular and humoral reaction is currently not at hand. The critical decision between the induction of type I and type II responses is made on the molecular level by the secretion of IL-12 or IL-10 from macrophages.

The fundamental decision by the immune system, whether a challenge is best addressed with a predominantly cellular response, a predominantly humoral response, or a combination of both, is at the heart of host integrity. Today, we know that not only the protection from infectious agents, but also the efficacy of vaccines, is dependent on the type of immune response elicited. In numerous cases, including vaccination against viruses, there is evidence to support the notion that combined type I and type II immunity may lead to improved vaccine success. Similarly, there is evidence to suggest that anti-HIV vaccinations could benefit from an adjuvant that elicits a combined Th1/Th2 response.

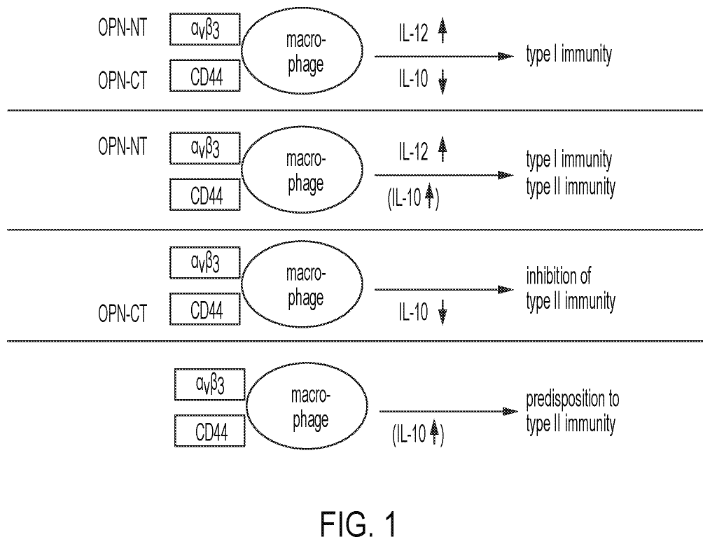

Regarding FIG. 1, Cellular (type I) and humoral (type II) immunity are regulated through distinct domains of OPN. Engagement of the integrin αᵥβ₃ on macrophages causes secretion of the type I cytokine IL-12, while interaction with CD44 causes suppression of the type II cytokine IL-10. In the absence of integrin αᵥβ₃ binding, IL-12 induction by other mechanisms is transient and inefficient. In the absence of CD44 engagement, type II cytokines are readily produced. As shown in the present disclosure, the selective, favorable influence of OPN-NT on Th1/Th2 cytokine production renders OPN-NT a promising vaccine adjuvant.

In embodiments, full-length human OPN comprises a nucleotide sequence having at least 80%, at least 85%, at least 90%, at least 95%, at least 98%, or at least 99% sequence identity to SEQ ID NO: 4. In a specific embodiment, the full-length human OPN DNA sequence comprises SEQ ID NO: 4. In embodiments, the full-length human OPN comprises a protein sequence having at least 80%, at least 85%, at least 90%, at least 95%, at least 98%, or at least 99% sequence identity to SEQ ID NO: 5. In a specific embodiment, the full-length human OPN protein sequence comprises SEQ ID NO: 5.

(SEQ ID NO: 5):
MRIAVICFCLLGITCAIPVKQADSGSSEEKQLYNKYPDAVATWLNPDPS
QKQNLLAPQNAVSSEETNDFKQETLPSKSNESHDHMDDMDDEDDDDHVD
SQDSIDSNDSDDVDDTDDSHQSDESHHSDESDELVTDFPTDLPATEVFT
PVVPTVDTYDGRGDSVVYGLRSKSKKFRRPDIQYPDATDEDITSHMESE
ELNGAYKAIPVAQDLNAPSDWDSRGKDSYETSQLDDQSAETHSHKQSRL
YKRKANDESNEHSDVIDSQELSKVSREFHSHEFHSHEDMLVVDPKSKEE
DKHLKFRISHELDSASSEVN.

In another embodiment, full-length murine OPN comprises a nucleotide sequence having at least 80%, at least 85%, at least 90%, at least 95%, at least 98%, or at least 99% sequence identity to SEQ ID NO: 2. In a specific embodiment, the full-length murine OPN DNA sequence comprises SEQ ID NO: 2. In embodiments, the full-length murine OPN comprises a protein sequence having at least 80%, at least 85%, at least 90%, at least 95%, at least 98%, or at least 99% sequence identity to SEQ ID NO: 3. In a specific embodiment, the full-length murine OPN protein sequence comprises SEQ ID NO: 3:

(SEQ ID NO: 3):
MRLQFSWLNSEGLTTTMRLAVICFCLFGIASSLPVKVTDSGSSEEKKLY
SLHPDPIATWLVPDPSQKQNLLAPQNAVSSEEKDDFKQETLPSNSNESH
DHMDDDDDDDDDDGDHAESEDSVDSDESDESHHSDESDETVTASTQADT
FTPIVPTVDVPNGRGDSLAYGLRSKSRSFQVSDEQYPDATDEDLTSHMK
SGESKESLDVIPVAQLLSMPSDQDNNGKGSHESSQLDEPSLETHRLEHS
KESQESADQSDVIDSQASSKASLEHQSHKFHSHKDKLVLDPKSKEDDRY
LKFRISHELESSSSEVN.

Accordingly, in one embodiment, a vaccine adjuvant comprising an N-terminal domain of osteopontin (OPN-NT) or a fragment thereof is provided. OPN-NT leverages the unique domain structure of osteopontin, which allows the separation of the IL-12-inducing domain from the IL-10-inhibiting domain. Elimination of the interaction by the C-terminus with its cognate receptor CD44 prevents IL-10 suppression and causes heightened immune responses, such as excessive granuloma formation. In embodiments, OPN-NT comprises an RGD domain, including the tri-peptide Arginine-Glycine-Aspartate (RGD). The RGD sequence has been identified as a motif for binding a subset of integrins, including αᵥβ3.

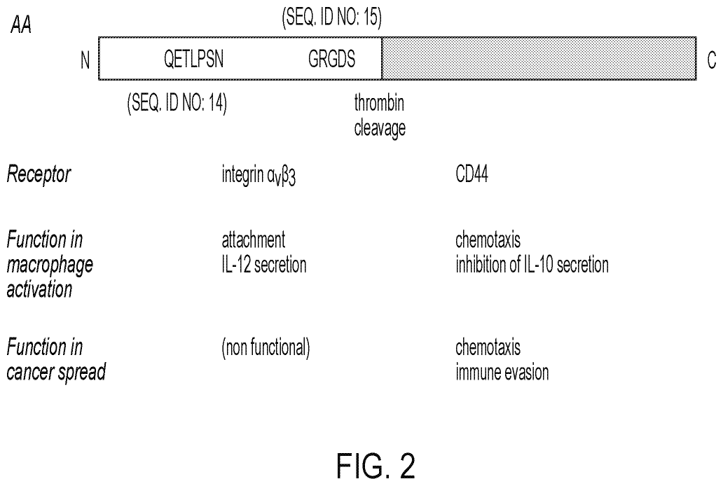

FIG. 2 depicts a map of OPN, wherein the N-terminal domain comprising an RGD domain (SEQ ID NO: 15) and an amino acid sequence QETLPSN (SEQ ID NO: 14), each of which confers activity to an OPN-NT protein or fragment thereof as described herein. FIG. 2 illustrates that the N-terminal portion of OPN binds integrin αᵥβ₃, and functions in attachment and IL-12 secretion of macrophages. The C-terminal portion of OPN, downstream of the thrombin cleavage site, binds to CD44 and functions in chemotaxis and inhibition of IL-10 secretion of macrophages. The C-terminal portion of OPN also functions in chemotaxis and immune evasion in cancer spread.

The OPN-NT sequence is selected to maximize the modulatory effects on Th1 and Th2 cytokines. In embodiments, the RGD sequence of OPN comprises amino acids 159-161 of SEQ ID NO: 1 (human), or amino acids 161-163 of SEQ ID NO: 3 (murine). In embodiments, OPN-NT comprises about the first 170 amino acids of the full-length OPN protein sequence. In embodiments, OPN-NT comprises the amino acid sequence upstream of a thrombin cleavage site between amino acids 168 and 169 of SEQ ID NO: 5, which serves to separate OPN-NT from OPN-CT. In a specific embodiment, human OPN-NT comprises a protein sequence having at least 80%, at least 85%, at least 90%, at least 95%, at least 98%, or at least 99% sequence identity to SEQ ID NO: 1. In a more specific embodiment, human OPN-NT comprises SEQ ID NO: 1:

(SEQ ID NO: 1):
MRIAVICFCLLGITCAIPVKQADSGSSEEKQLYNKYPDAVATWLNPDPS
QKQNLLAPQNAVSSEETNDFKQETLPSKSNESHDHMDDMDDEDDDDHVD
SQDSIDSNDSDDVDDTDDSHQSDESHHSDESDELVTDFPTDLPATEVFT
PVVPTVDTYDGRGDSVVYGLR

wherein the secretion signal sequence is underlined. In embodiments, SEQ ID NO: 1 is encoded by a nucleic acid comprising a stop codon after the last residue of the OPN-NT sequence. In a specific embodiment, the nucleotide encoding OPN-NT has at least 80%, at least 85%, at least 90%, at least 95%, at least 98%, or at least 99% sequence identity to SEQ ID NO: 19.

In embodiments, the vaccine adjuvant comprises a fragment of OPN-NT. The skilled artisan will appreciate that the fragment of OPN-NT is a truncated and/or engineered construct of OPN-NT that maintains the functionality of OPN-NT with respect to its modulatory effect on Th1/Th2 cytokine production. In embodiments, the OPN-NT sequence is truncated by removal of exon 4, exon 5, or both exon 4 and exon 5.

In another embodiment, murine OPN-NT comprises a protein sequence having at least 80%, at least 85%, at least 90%, at least 95%, at least 98%, or at least 99% sequence identity to SEQ ID NO: 9, SEQ ID NO: 10, or SEQ ID NO: 11. In embodiments, murine OPN-NT comprises:

(SEQ ID NO: 9):
MRLQFSWLNSEGLTTTMRLAVICFCLFGIASSLPVKVTDSGSSEEKKLY
SLHPDPIATWLVPDPSQKQNLLAPQNAVSSEEKDDFKQETLPSNSNESH
DHMDDDDDDDDDDGDHAESEDSVDSDESDESHHSDESDETVTASTQADT
FTPIVPTVDVPNGRGDSLAYGLR;
(SEQ ID NO: 10):
MRLQFSWLNSEGLTTTMRLAVICFCLFGIASSQETLPSNSNESHDHMDD
DDDDDDDDGDHAESEDSVDSDESDESHHSDESDETVTASTQADTFTPIV
PTVDVPNGRGDSLAYGLR;
or
(SEQ ID NO: 11):
MRLQFSWLNSEGLTTTMRLAVICFCLFGIASSLPVKVTDSGSSEEKQNA
VSSEEKDDFKQETLPSNSNESHDHMDDDDDDDDDDGDHAESEDSVDSDE
SDESHHSDESDETVTASTQADTFTPIVPTVDVPNGRGDSLAYGLR.

In embodiments, SEQ ID NOs: 9-11 are encoded by nucleic acids comprising a stop codon after the last residue of each OPN-NT sequence.

SEQ ID NO: 10 corresponds to a truncated amino acid sequence of OPN-NT, wherein amino acids 33-86 of SEQ ID NO: 9 are deleted from the sequence. SEQ ID NO: 11 corresponds to a truncated amino acid sequence of OPN-NT, wherein exon 4 has been deleted from the sequence. Illustratively, SEQ ID NOs: 10 and 11 represent fragments of murine OPN-NT suitable for use in the adjuvants, fusion proteins, conjugates, vaccines, and methods of the present disclosure.

In embodiments, human OPN-NT comprises a protein sequence having at least 80%, at least 85%, at least 90%, at least 95%, at least 98%, or at least 99% sequence identity to SEQ ID NO: 20, SEQ ID NO: 21, or SEQ ID NO: 22. In embodiments, human OPN-NT comprises:

(SEQ ID NO: 20):
MRIAVICFCLLGITCAIPVKQADSGSSEEKQLYNKYPDAVATWLNPDPS
QKQNLLAPQTLPSKSNESHDHMDDMDDEDDDDHVDSQDSIDSNDSDDVD
DTDDSHQSDESHHSDESDELVTDFPTDLPATEVFTPVVPTVDTYDGRGD
SVVYGLR;
(SEQ ID NO: 21):
MRIAVICFCLLGITCAIPVKQADSGSSEEKQNAVSSEETNDFKQETLPS
KSNESHDHMDDMDDEDDDDHVDSQDSIDSNDSDDVDDTDDSHQSDESHH
SDESDELVTDFPTDLPATEVFTPVVPTVDTYDGRGDSVVYGLR;
or
(SEQ ID NO: 22):
MRIAVICFCLLGITCAIPVKQADSGSSEEKQTLPSKSNESHDHMDDMDD
EDDDDHVDSQDSIDSNDSDDVDDTDDSHQSDESHHSDESDELVTDFPTD
LPATEVFTPVVPTVDTYDGRGDSVVYGLR.

SEQ ID NO: 20 corresponds to a truncated amino acid sequence of human OPN-NT, wherein exon 5 has been deleted from the sequence. SEQ ID NO: 21 corresponds to a truncated amino acid sequence of human OPN-NT, wherein exon 4 has been deleted from the sequence. SEQ ID NO: 21 corresponds to a truncated amino acid sequence of human OPN-NT, wherein both exon 4 and exon 5 have been deleted from the sequence. Illustratively, SEQ ID NOs: 20-22 represent fragments of human OPN-NT suitable for use in the adjuvants, fusion proteins, conjugates, vaccines, and methods of the present disclosure.

In embodiments, OPN-NT comprises a signal sequence for facilitating secretion of the N-terminal OPN domain by a cell. The signal sequence of OPN is a sequence comprising about the first 16-17 amino acids of SEQ ID NO: 1 (human) or SEQ ID NO: 3 (murine). In embodiments, the signal sequence has at least 80%, at least 85%, at least 90%, at least 95%, at least 98%, or at least 99% sequence identity to SEQ ID NO: 18. In a specific embodiment, the signal sequence of human OPN comprises the amino acid sequence MRIAVICFCLLGITCAI (SEQ ID NO: 18).

In embodiments, OPN-NT is administered with and/or conjugated to an immunizing antigen. An immunizing antigen, or immunogen, is a substance that generates an adaptive (type I and/or type II) immune response in a host organism. In embodiments, an immunizing antigen may stimulate the production of antibodies and/or stimulate a T-cell response by the host immune system, without inducing a disease state in the host. Suitable immunizing antigens include, but are not limited to, a protein or immunizing fragment thereof, a nucleic acid, a virus, a pseudovirus, a bacterium, or a parasite.

As used herein, “pseudovirus” refers to a synthetic chimera comprising a surrogate viral core derived from a parent virus and an envelope glycoprotein derived from a heterologous virus. Typically, the parent viral genome is modified to delete essential genes required for replication. Optionally, a reporter gene coding for luciferase or a fluorescent protein is inserted into the pseudovirus genome, which facilitates quantification of gene expression. Pseudoviruses are only capable of undergoing a single infection cycle in a host, but permit study of viral entry mechanisms.

Optionally, the immunizing antigen is an inactivated or attenuated microorganism, such as an inactivated or attenuated virus, pseudovirus, bacterium, or parasite. A microorganism or parasite may be inactivated (e.g., killed) chemically, for example, by contacting the microorganism with formaldehyde, or by applying heat to the microorganism or parasite. An attenuated microorganism or parasite is a viable microorganism or parasite having reduced virulence, often generated via serial passage or chemical modification. The skilled artisan will appreciate that various methods of inactivating or attenuating a microorganism or parasite are well known in the art and suitable for use in the present disclosure.

In embodiments, the immunizing antigen is an inactivated parasite or an immunogenic component thereof. In a specific embodiment, the parasite is selected from the group consisting of hookworms, liver flukes, Trypanosoma, Plasmodium spp., Schistosoma, and the like. In a specific embodiment, the immunizing antigen is a parasite wall component, such as a merozoite coat of Plasmodium, a variant surface glycoprotein (VSG) coat of Trypanosoma, and the like.

In another embodiment, the immunizing antigen is an attenuated or inactivated bacterium or an immunogenic component thereof. In a specific embodiment, the bacterium is selected from the group consisting of Mycobacterium tuberculosis, Borrelia burgdorferi, Bacillus anthracis, Enterococcus faecium, Staphylococcus aureus, Klebsiella pneumoniae, Acinetobacter baumanii, Pseudomonas aeruginosa, Enterobacter spp., Clostridioides difficile, Vibrio cholera, Clostridium tetani, Corynebacterium diphtheria, Salmonella spp., Haemophilus influenza type B, Yersinia pestis, Listeria monocytogenes, Shigella spp., Mycobacterium bovis, and the like. In a specific embodiment, the immunizing antigen is an immunogenic component of a bacterium, including but not limited to, proteins, peptides, nucleic acids, toxins, toxoids, polysaccharides, lipopolysaccharides, flagella, adhesins, outer membrane components, cell wall components, subunits thereof, and the like.

In another specific embodiment, the immunizing antigen is an inactivated virus or an immunogenic component thereof. In a specific embodiment, the virus is selected from the group consisting of influenza, SARS-CoV, SARS-CoV-2, Middle East Respiratory Syndrome virus (MERS), human immunodeficiency virus (HIV), respiratory syncytial virus (RSV), Dengue virus (DV), Cytomegalovirus (CMV), Zika virus, Ebola virus, West Nile virus, Yellow Fever virus, Japanese encephalitis virus, Chikungunya virus, and the like. In a more specific embodiment, the immunizing antigen is an inactivated influenza virus. In another specific embodiment, the immunizing antigen is an immunogenic component of a virus, including but not limited to, proteins, peptides, nucleic acids, viral membranes, viral subunits, and the like.

As used herein with respect to immunizing antigens, the term “subunit” refers to a purified portion of a whole pathogen (virus, bacterium, parasite, etc.), which has the capacity to trigger an immune response in a host, but which is incapable of causing disease in the host. Subunit vaccine may include protein subunits, polysaccharide subunits, or conjugate subunits comprising a subunit bound to a carrier moiety.

In another embodiment, the immunizing antigen is a protein or immunogenic fragment thereof obtained or derived from a pathogen, such as a virus, a bacterium, or a parasite. In a more specific embodiment, the immunizing antigen is a viral, bacterial, or parasitic protein or fragment thereof. In a very specific embodiment, the immunizing antigen is a viral protein or fragment thereof derived from severe acute respiratory syndrome coronavirus type 2 (SARS-CoV-2). SARS-CoV-2 contains various proteins that may serve as effective immunizing antigens, including spike (S) glycoprotein, which binds with the ACE-2 receptor of host cells and facilitates viral entry. In a very specific embodiment, the immunizing antigen is a receptor binding domain of the SARS-CoV-2 spike glycoprotein, illustratively set forth in SEQ ID NO: 6. Optionally, the immunizing antigen has at least 80%, at least 85%, at least 90%, at least 95%, at least 98%, or at least 99% sequence identity to SEQ ID NO: 6. SEQ ID NO: 6 comprises a 7-histidine tag (underlined) which optionally may be cleaved or deleted from the sequence for use in the constructs and methods disclosed herein.

(SEQ ID NO: 6):
LLKYNENGTITDAVDCALDPLSETKCTLKSFTVEKGIYQTSNFRVQPTE
SIVRFPNITNLCPFGEVFNATRFASVYAWNRKRISNCVADYSVLYNSAS
FSTFKCYGVSPTKLNDLCFTNVYADSFVIRGDEVRQIAPGQTGKIADYN
YKLPDDFTGCVIAWNSNNLDSKVGGNYNYLYRLFRKSNLKPFERDISTE
IYQAGSTPCNGVEGFNCYFPLQSYGFQPTNGVGYQPYRVVVLSFELLHA
PATVCGPKKSTNGSGHHHHHHH.

In another embodiment, a fusion protein or a nucleic acid construct encoding a fusion protein is provided, the fusion protein comprising OPN-NT or a fragment thereof conjugated to a protein or fragment thereof derived from a pathogen, such as a virus, a bacterium, or a parasite. In a specific embodiment, the pathogenic protein or fragment thereof is derived from SARS-CoV-2. Optionally, the SARS-CoV-2 protein is a spike glycoprotein having at least 80%, at least 85%, at least 90%, at least 95%, at least 98%, or at least 99% sequence identity to SEQ ID NO: 6. In a more specific embodiment, the fusion protein comprises an amino acid sequence having at least 80%, at least 85%, at least 90%, at least 95%, at least 98%, or at least 99% sequence identity to SEQ ID NO: 7. In a very specific embodiment, the fusion protein comprises SEQ ID NO: 7. SEQ ID NO: 7 comprises a 7-histidine tag (underlined) which optionally may be cleaved or deleted from the sequence for use in the constructs and methods disclosed herein.

(SEQ ID NO: 7):
MRIAVICFCLLGITCAIPVKQADSGSSEEKQLYNKYPDAVATWLNPDPS
QKQNLLAPQNAVSSEETNDFKQETLPSKSNESHDHMDDMDDEDDDDHVD
SQDSIDSNDSDDVDDTDDSHQSDESHHSDESDELVTDFPTDLPATEVFT
PVVPTVDTYDGRGDSVVYGLRLLKYNENGTITDAVDCALDPLSETKCTL
KSFTVEKGIYQTSNFRVQPTESIVRFPNITNLCPFGEVENATRFASVYA
WNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQP
TNGVGYQPYRVVVLSFELLHAPATVCGPKKSTNGSGHHHHHHH.

### Methods of Use

A method for potentiating an immune response to an immunizing antigen in a subject is also provided, the method comprising administering to the subject an effective amount of a vaccine adjuvant comprising OPN-NT or a fragment thereof, according to any of the embodiments disclosed herein.

The term “effective amount,” as used herein, refers to the amount of a composition that is sufficient to achieve a desired biological effect. Generally, the dosage needed to provide an effective amount of the composition will vary depending upon such factors as the subject's age, condition, sex, and other variables which can be adjusted by one of ordinary skill in the art. The compositions of the present disclosure can be administered by either single or multiple dosages of an effective amount. In a specific embodiment, the effective amount is an amount sufficient to elicit a combined Th1/Th2 adaptive immune response in the subject.

In embodiments, the vaccine adjuvant is co-administered with an immunizing antigen according to any of the embodiments disclosed herein. “Co-administered,” as used herein, refers to administration of the adjuvant and the immunizing antigen such that both agents can simultaneously achieve a physiological effect, e.g., in a recipient subject. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the subject. Thus, in embodiments, the adjuvant and the immunizing antigen may be administered concurrently or sequentially.

In certain embodiments, the methods disclosed herein comprise administering to the subject an effective amount of an OPN-NT adjuvant according to any of the embodiments disclosed herein, conjugated to the immunizing antigen. In embodiments, the OPN-NT-immunogen conjugate is a fusion protein construct, a nucleic acid construct, or a conjugate of OPN-NT and an inactivated or attenuated pathogen or component thereof, according to any of the embodiments disclosed herein.

In a specific embodiment, the OPN-NT adjuvant is transduced as a nucleic acid construct expressed by a cell, i.e., a cell of the subject. In such embodiments, the nucleic acid construct may be a DNA or RNA construct encoding a fusion protein according to any of the embodiments disclosed herein. In a specific embodiment, the nucleic acid construct has at least 80%, at least 85%, at least 90%, at least 95%, at least 98%, or at least 99% sequence identity to SEQ ID NO: 8. In a more specific embodiment, the nucleic acid construct is a DNA or RNA construct comprising SEQ ID NO: 8.

In another embodiment, a cell engineered to express an OPN-NT adjuvant or OPN-NT fusion protein according to any embodiments of the present disclosure is provided.

In another embodiment, a nucleic acid encoding an OPN-NT fusion protein according to any embodiments of the present disclosure is provided.

In yet another embodiment, a vector, such as a viral vector, comprising an OPN-NT fusion protein according to any embodiments of the present disclosure is provided. Various suitable viral vectors are known in the art, including but not limited to adenovirus, adeno-associated virus (AAV), herpes virus, retroviruses, and the like.

Also provided herein is a method for vaccinating a subject against SARS-CoV-2, the method comprising administering to the subject an effective amount of a fusion protein comprising OPN-NT according to any of the embodiments disclosed herein and a pathogenic protein or fragment thereof derived from SARS-CoV-2. Optionally, the SARS-CoV-2 protein is a spike glycoprotein, or more particularly, the SARS-CoV-2 protein is an ACE-2 receptor binding domain of the spike glycoprotein. In embodiments, the fusion protein comprises an amino acid sequence having at least 80%, at least 85%, at least 90%, at least 95%, at least 98%, or at least 99% sequence identity to SEQ ID NO: 7. In a very specific embodiment, the fusion protein comprises SEQ ID NO: 7.

A cellular vaccine is also provided, comprising a cell engineered to express an OPN-NT adjuvant and a pharmaceutically acceptable carrier. In embodiments, the cell is an autologous cell obtained from the subject who will receive the vaccine.

The pharmaceutically acceptable carrier, or excipient, must be “acceptable” in the sense of being compatible with the other ingredients of the vaccine formulation and not deleterious to the recipients thereof. The disclosure further includes a vaccine composition, in combination with packaging material suitable for the vaccine composition, including instructions for the use of the composition in vaccination of subjects in need thereof.

Vaccine compositions include those suitable for parenteral administration. In a specific embodiment, the compositions disclosed herein are suitable for intramuscular administration, although other specific means of parenteral administration are also viable (such as, for example, intravenous, intra-arterial, or subcutaneous administration). The compositions may be prepared by any methods well known in the art of pharmacy, for example, using methods such as those described in Remington: The Science and Practice of Pharmacy (21st ed., Lippincott Williams and Wilkins, 2005, see Part 5: Pharmaceutical Manufacturing). Suitable pharmaceutical carriers are well-known in the art. See, for example, Handbook of Pharmaceutical Excipients, Sixth Edition, edited by Raymond C. Rowe (2009). The skilled artisan will appreciate that certain carriers may be more desirable or suitable for certain modes of administration of an active ingredient. It is within the purview of the skilled artisan to select the appropriate carriers for a given vaccine composition.

For parenteral administration, suitable compositions include aqueous and non-aqueous sterile suspensions for intramuscular and/or intravenous administration. The compositions may be presented in unit dose or multi-dose containers, for example, sealed vials and ampoules.

As will be understood by those of skill in this art, the specific dose level for any particular subject will depend on a variety of factors, including the activity of the agent employed; the age, body weight, general health, and sex of the individual being treated; the time and route of administration; the rate of excretion; and the like.

In another embodiment, a method of vaccinating a subject in need thereof is provided, the method comprising: obtaining autologous cells from the subject; transducing the autologous cells with a nucleic acid encoding OPN-NT; and reintroducing the autologous cells into the subject. Optionally, the nucleic acid encoding OPN-NT encodes a fusion protein comprising OPN-NT and an immunizing antigen as disclosed herein.

Advantageously, the adjuvants, fusion proteins, conjugates, and vaccines disclosed herein induce a combined Th1 and Th2 adaptive immune response in the subject to whom the agent(s) are administered. Further, the disclosed OPN-NT adjuvants potentiate the host immune response to the immunizing antigen.

### EXAMPLES

The following examples are given by way of illustration are not intended to limit the scope of the disclosure.

### Example 1. Adjuvant Structure and Preparation

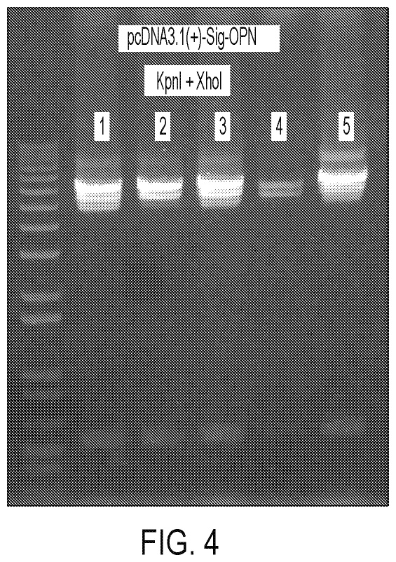

Osteopontin comprising a signal sequence for secretion was subcloned into pcDNA3.1(+) at the KpnI/XhoI sites, as shown in FIG. 4. Clone 1 was selected and confirmed by sequencing and named pcDNA3.1(+)-Sig-OPN.

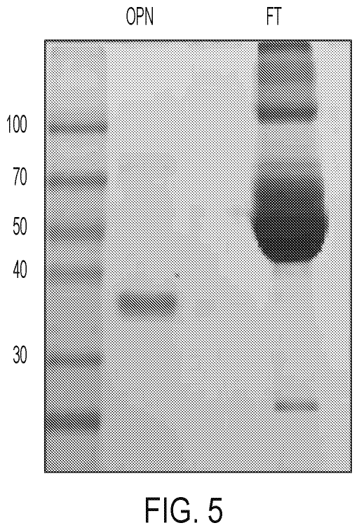

The construct was transiently transfected into HEK293 cells for protein expression and purification. The medium from transfected cells was collected for protein purification on nickel columns. Purified OPN from the nickel column and the column flow-through were characterized by gel electrophoresis, as shown in FIG. 5. Eluted protein from the transient transfection was exchanged into phosphate buffered saline (PBS) and concentrated.

### Example 2. Synthesis of Recombinant Adenoviruses Expressing Fusion Protein of OPN and SARS-CoV-2 Spike Protein (OPN-COV)

Nucleotide sequences comprising OPN and SARS-CoV-2 spike protein were subcloned into entry vector pShuttle-CMV at KpnI/XhoI restriction sites to generate a recombinant adenovirus construct. The insert was confirmed by sequencing. A tag of 7×His was placed at the C-terminus for affinity purification (pShuttle-OPNCOV-6). Another version of the OPN-COV without the secretion signal sequence was also cloned into pShuttle-CMV vector for purification protein from cell pellet (pShuttle-OPNCOV-PCR-4).

Both entry plasmids pShuttle-OPNCOV-6 and pShuttle-OPNCOV-PCR-4 were linearized with PmeI single digestion and transformed into competent BJ5183 cells with adenovirus backbone plasmid pAdEasy-1.

After in vivo recombination, colonies containing recombinant virus genome were selected, linearized with PacI single digestion, and then transfected into packaging HEK293 cells following established protocols.

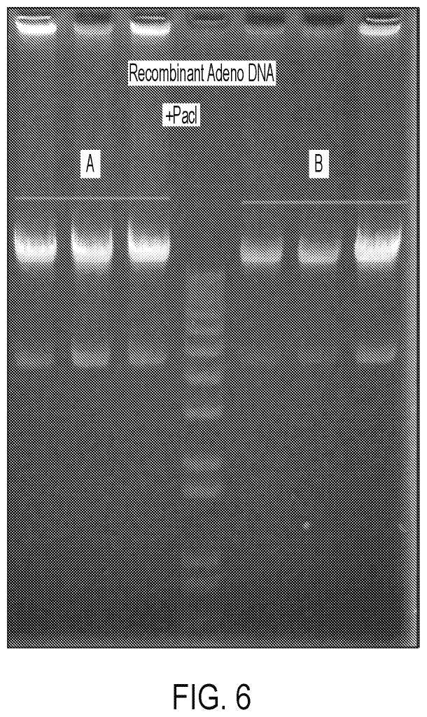

Live recombinant adenoviruses harboring both cassettes (i.e., with and without the secretion signal) were generated for protein expression and purification. Results of the PacI digestion are shown in FIG. 6, wherein bands for the signal-containing OPN-COV (A lanes) and OPN-COV without a signal sequence (B lanes) are evident at 4.5 kb (cassettes) and >30 kb (vector).

### Example 3. pcDNA3.1 Vector Based Transient Transfection to Produce OPN-COV Protein in HEK293 Cells

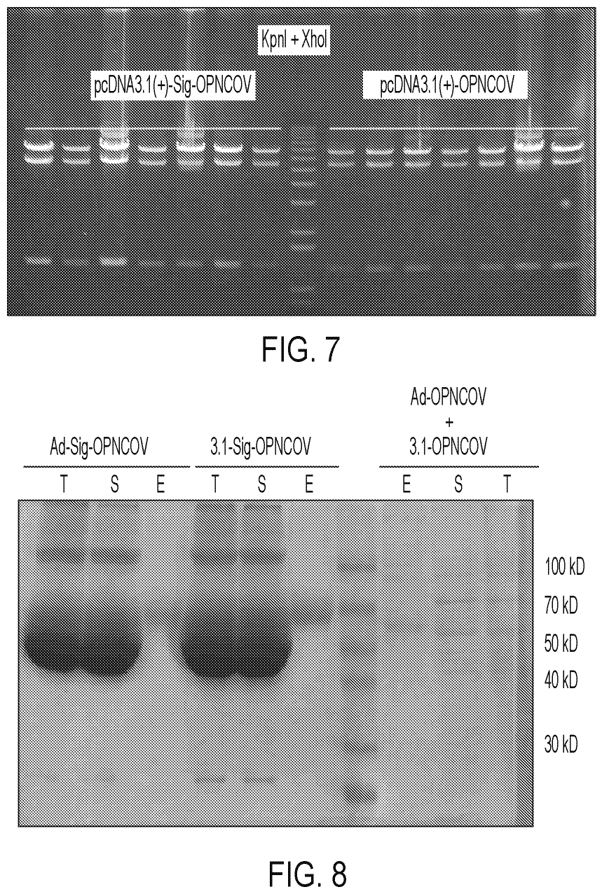

In order to transiently express protein in HEK293 cells, OPN-COV constructs with and without signal sequence were retrieved with KpnI+XhoI from the confirmed pShuttle constructs described in Example 2 and subcloned into pcDNA3.1(+) at KpnI/XhoI site. Validation of the constructs is shown in FIG. 7, wherein the 7 left-hand lanes comprise pcDNA3.1(+)-Sig-OPNCOV (with signal sequence) and the 7 right-hand lanes comprise pcDNA3.1(+)-OPNCOV (without signal sequence). After KpnI/XhoI digestion, bands are evident at 1.3 kb and 5.4 kb for pcDNA3.1(+)-Sig-OPNCOV and at 1.25 kb and 5.4 kb for pcDNA3.1(+)-OPNCOV.

### Example 4. OPN-COV Protein Expression and Purification

The recombinant OPN-COV adenoviruses were infected into HEK293 cells and the pcDNA3.1 constructs were transiently transfected into HEK293 cells for protein expression and purification analyses. For the signal-containing constructs, culture medium was collected for analysis. For non-signal-containing constructs, cell pellets were harvested for protein purification on nickel columns.

Results are shown in FIG. 8, which is an image of an SDS-PAGE gel showing total protein (T), medium supernatant (S), elution nickel column (E) fractions for medium supernatant from signal-containing adenovirus infected HEK293 cells (Ad-Sig-OPNCOV), medium supernatant from transient transfection with pcDNA3.1 construct with signal-containing OPNCOV (3.1Sig-OPNCOV), and combined cell pellets from Ad-OPNCOV-infected cells and transiently infected cells by pcDNA3.1-OPNCOV, both without signal sequence (Ad-OPNCOV+3.1-OPNCOV). Results show that both adenovirus and transient transfection of OPNCOV constructs comprising the signal sequence yielded successfully secreted fusion protein into the culture medium. The eluted fusion protein from transient transfection was exchanged into PBS buffer and concentrated for further analysis.

### Example 5. Conjugation of OPN-NT to a Virus Particle

Vaccination will be carried out intramuscularly with 45 μg of 0.74% formaldehyde-inactivated A/Puerto Rico/8/1934 [H1N1] (PR8) virus mixed with 10 μg of OPN-NT. For the IPR8-R848 conjugate vaccine, an amine derivative of OPN-NT is linked to SM(PEG)4 by incubation in DMSO for 24 hr at 37° C. OPN-NT-SM(PEG)4 will then be incubated with influenza virus that has been reduced to generate free thiol groups (IPR8-OPN-NT). Unconjugated OPN-NT will be removed by extensive dialysis. This construct will then be inactivated by treatment with 0.74% formaldehyde for 1 hr at 37° C., followed by dialysis. Successful conjugation will be assessed by differential stimulation of RAW264.7 cells consecutive to incubation with similar doses (based on protein content) of OPN-NT-conjugated versus non-conjugated vaccine. Endotoxin and nucleic acids will be removed using an Acrodisc Mustang Q capsule and purified proteins are extensively dialyzed against PBS.

Expected results will show that the inactivated H1N1 virus conjugate comprises on its surface a plurality of OPN-NT proteins or fragments thereof, which are tightly attached.

### Example 6. Animal Studies

Animal subjects will be vaccinated intramuscularly with 45 μg of 0.74% formaldehyde-inactivated A/Puerto Rico/8/1934 [H1N1] (PR8) virus mixed with either 100 μg of the N-terminal osteopontin domain (n=4) or no admixture (n=4). Control animals will receive PBS (n=2). All injections will be delivered intramuscularly into the deltoid muscle (500 μl volume). The animals will receive the test composition in the right arm and PBS in the left arm.

Expected results include a strong induction of antibody and cytotoxic T-cell production to the immunizing antigen, which exceeds both the antibody levels and cytotoxic T-cell activities achievable with adjuvant-free antigen alone. The positive results will be measurable as antibody titers and in ex vivo CTL assays. The combined Th1/Th2 response elicited by the adjuvant, being measurable according to the levels of relevant cytokines in the blood or their RNA messages in the lymph nodes, will enhance memory formation in both the B-cell compartment and the T-cell compartment. Because the predominant effector cells are cytotoxic T-lymphocytes, the induction of CTL activity against antigen-bearing cells will be reflected in cytotoxicity assays after enrichment of CD8+ cells from post-vaccination lymph nodes with antibody-coupled magnetic beads. Effector-to-target ratios range from 0.1 to 100.

The elicitation of serum antibodies to the PR8 vaccine will be tested in solid phase ELISA, where the inactivated virus is immobilized on the plate to be probed by serum titration. Choice of secondary antibody permits probing for specific immunoglobulin isotypes. Osteopontin has been described to induce B-cell activation and IgM/IgG secretion in vitro and in vivo.

### Example 7. Autologous Cells as Vaccines

The use of autologous cells as vaccines to augment anti-tumor immunity has had good success when these cells were genetically modified to express certain cytokines. The transfection of various cytokines into these cells has proven efficacious in generating cellular anti-tumor vaccines.

The full length osteopontin gene was transduced into B16-F10 murine melanoma cells and the protective effect of irradiated transfectants against challenge with untransduced B16-F10 cells was assessed. The osteopontin vaccine roughly doubled the survival time after tumor challenge. Comparable levels of protection have been observed before for other vaccines based on type I cytokines, such as IL-12 and IL-2. It was thus hypothesized that OPN-NT would induce combined type I and type II immunity and may completely protect from challenge. An OPN-NT construct was transduced into B16-F10 cells. Protection was more complete with the OPN-NT vaccine.

### Example 8. Generation of Murine OPN Constructs

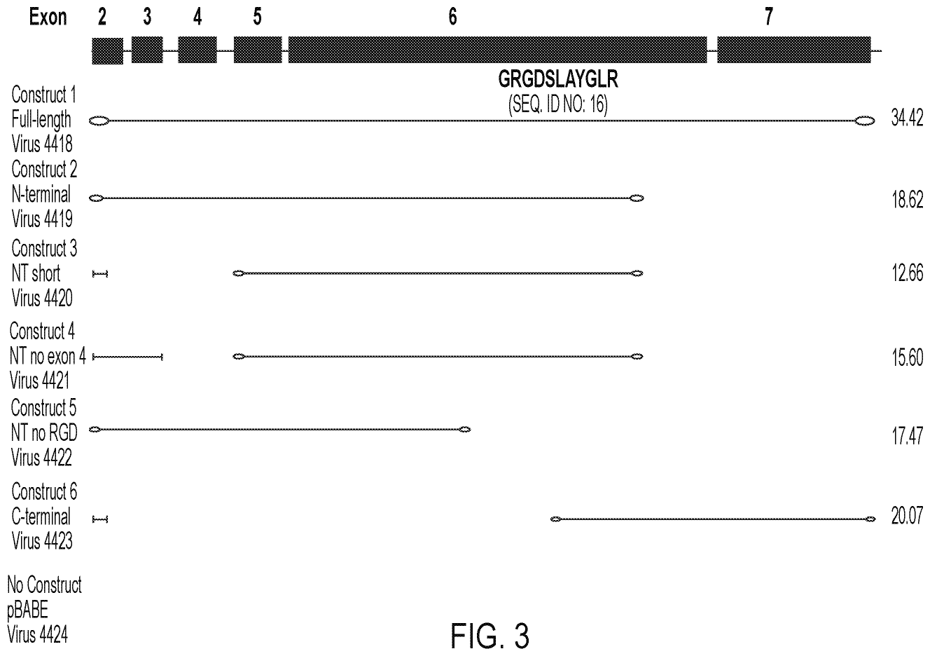

FIG. 3 depicts a map of murine OPN exons and various protein constructs that were synthesized therefrom. Construct 1 depicts a full-length murine OPN protein, corresponding to SEQ ID NO: 3. Construct 2 depicts an N-terminal murine OPN domain (OPN-NT), corresponding to SEQ ID NO: 9. Construct 3 depicts an N-terminal murine OPN domain corresponding to SEQ ID NO: 10, wherein amino acids 33-86 are deleted relative to SEQ ID NO: 9. Construct 4 depicts an N-terminal OPN domain corresponding to SEQ ID NO: 11, wherein exon 4 is deleted relative to SEQ ID NO: 9. Construct 5 depicts an N-terminal murine OPN domain corresponding to SEQ ID NO: 12, wherein the RGD domain (SEQ ID NO 16) has been deleted. Construct 6 depicts a C-terminal murine OPN domain, comprising a signal sequence, corresponding to SEQ ID NO: 13.

Aspects of the present disclosure can be described with reference to the following numbered clauses, with preferred features laid out in dependent clauses.

1. A vaccine adjuvant comprising an N-terminal domain of osteopontin or a fragment thereof.

2. The vaccine adjuvant according to clause 1, wherein the N-terminal domain of osteopontin or the fragment thereof comprises a secretion signal sequence.

3. The vaccine adjuvant according to any of the preceding clauses, wherein exon 4, exon 5, or both exon 4 and exon 5 of osteopontin are not present in the N-terminal domain of osteopontin.

4. The vaccine adjuvant according to any of the preceding clauses, wherein the N-terminal domain of osteopontin or the fragment thereof is conjugated to an immunizing antigen.

5. The vaccine adjuvant according to clause 4, wherein the immunizing antigen is a protein or fragment thereof, a nucleic acid, a virus, a pseudovirus, a bacterium, or a parasite.

6. The vaccine adjuvant according to clause 4 or clause 5, wherein the immunizing antigen is an inactivated or attenuated virus, pseudovirus, bacterium, or parasite.

7. The vaccine adjuvant according to any of clauses 4-6, wherein the immunizing antigen is inactivated influenza virus.

8. The vaccine adjuvant according to any of clauses 4-6, wherein the immunizing antigen is a viral, bacterial, or parasitic protein or a subunit or fragment thereof.

9. The vaccine adjuvant according to clause 8, wherein the viral protein or fragment thereof is derived from SARS-CoV-2.

10. The vaccine adjuvant according to any of the preceding clauses, wherein the N-terminal domain of osteopontin comprises an amino sequence having at least 80% sequence identity with SEQ ID NO: 1.

11. The vaccine adjuvant according to any of the preceding clauses, wherein the N-terminal domain of osteopontin comprises SEQ ID NO: 1, SEQ ID NO. 20, SEQ ID NO: 21, or SEQ ID NO: 22.

12. A fusion protein comprising an N-terminal domain of osteopontin or a fragment thereof conjugated to an immunogenic protein or fragment thereof derived from a pathogenic virus.

13. The fusion protein according to clause 12, wherein the pathogenic virus is SARS-CoV-2.

14. The fusion protein according to clause 13, wherein the fusion protein comprises an amino acid sequence having at least 80% sequence identity with SEQ ID NO: 7.

15. The fusion protein according to any of clauses 12-14, wherein the fusion protein comprises SEQ ID NO: 7.

16. A cell engineered to express the fusion protein of any of clauses 12-15.

17. A nucleic acid encoding the fusion protein of any of clauses 12-15.

18. A vector comprising the nucleic acid of clause 17.

19. A method for potentiating an immune response to an immunizing antigen in a subject, the method comprising administering to the subject an effective amount of a vaccine adjuvant comprising an N-terminal domain of osteopontin or a fragment thereof.

20. The method according to clause 19, wherein the N-terminal domain of osteopontin or the fragment thereof comprises a signal sequence.

21. The method according to any of clauses 19-20, wherein exon 4, exon 5, or both exon 4 and exon 5 of osteopontin are not present in the N-terminal domain of osteopontin.

22. The method according to any of clauses 19-21, wherein the vaccine adjuvant is co-administered with the immunizing antigen.

23. The method according to any of clauses 19-22, wherein the immunizing antigen is a protein or fragment thereof, a nucleic acid, a virus, a pseudovirus, a bacterium, or a parasite.

24. The method according to any of clauses 19-22, wherein the immunizing antigen is an inactivated or attenuated virus, pseudovirus, bacterium, or parasite.

25. The method according to any of clauses 19-22, wherein the immunizing antigen is a viral, bacterial, or parasitic protein or fragment thereof.

26. The method according to any of clauses 19-25, wherein the N-terminal domain of osteopontin or the fragment thereof and the immunizing antigen are administered concurrently or sequentially.

27. The method according to any of clauses 19-25, wherein the N-terminal domain of osteopontin or the fragment thereof is conjugated to the immunizing antigen.

28. A method for vaccinating a subject against SARS-CoV-2, the method comprising administering to the subject an effective amount of the fusion protein of any of clauses 12-15.

29. A method of vaccinating a subject against SARS-CoV-2, the method comprising administering to the subject an effective amount of a fusion protein comprising an N-terminal domain of osteopontin or a fragment thereof and a receptor binding domain of SARS-CoV-2 spike glycoprotein.

30. The method according to clause 29, wherein the fusion protein has at least 80% sequence identity with SEQ ID NO: 7.

31. The method according to any of clauses 29-30, wherein the fusion protein comprises SEQ ID NO: 7.

32. The method according to any of clauses 19-27, wherein administration of the vaccine adjuvant induces a combined Th1 and Th2 adaptive immune response in the subject.

33. The method according to clauses 28-31, wherein administration of the fusion protein induces a combined Th1 and Th2 adaptive immune response in the subject.

34. A cell engineered to express the vaccine adjuvant of any of clauses 1-9.

35. A vaccine comprising:
the cell according to clause 34; and
a pharmaceutically acceptable carrier.

36. The vaccine according to clause 35, wherein the cell is an autologous cell obtained from a subject.

37. A method of vaccinating a subject in need thereof, the method comprising: obtaining autologous cells from the subject;
transducing the autologous cells with a nucleic acid encoding an N-terminal domain of osteopontin; and
reintroducing the autologous cells into the subject.

38. The method according to clause 37, wherein the N-terminal domain of osteopontin is present as part of a fusion protein comprising the N-terminal domain of osteopontin and an immunizing antigen.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

The foregoing description is illustrative of particular embodiments of the invention but is not meant to be a limitation upon the practice thereof. While particular embodiments have been illustrated and described, it would be obvious to one skilled in the art that various other changes and modifications can be made without departing from the spirit and scope of the invention. It is therefore intended to cover in the appended claims all such changes and modifications that are within the scope of this invention.

1. A vaccine adjuvant comprising an N-terminal domain of osteopontin or a fragment thereof, wherein exon 4, exon 5, or both exon 4 and exon 5 are not present in the N-terminal domain of osteopontin or the fragment thereof.

2. The vaccine adjuvant according to claim 1, wherein the N-terminal domain of osteopontin or the fragment thereof comprises a secretion signal sequence.

3. (canceled)

4. The vaccine adjuvant according to claim 2, wherein the N-terminal domain of osteopontin or the fragment thereof is conjugated to an immunizing antigen selected from the group consisting of a protein or fragment thereof, a nucleic acid, a virus, a pseudovirus, a bacterium, or a parasite, wherein the virus, the pseudovirus, the bacterium or the parasite is inactivated or attenuated.

5-6. (canceled)

7. The vaccine adjuvant according to claim 4, wherein the immunizing antigen is inactivated influenza virus.

8. The vaccine adjuvant according to claim 4, wherein the immunizing antigen is a viral, bacterial, or parasitic protein or a subunit or fragment thereof.

9. The vaccine adjuvant according to claim 8, wherein the viral protein or fragment thereof is derived from SARS-CoV-2.

10. The vaccine adjuvant according to claim 1, wherein the N-terminal domain of osteopontin comprises an amino sequence having at least 80% sequence identity with SEQ ID NO: 1.

11. The vaccine adjuvant according to claim 1, wherein the N-terminal domain of osteopontin comprises SEQ ID NO: 1, SEQ ID NO. 20, SEQ ID NO: 21, or SEQ ID NO: 22.

12. A fusion protein comprising an N-terminal domain of osteopontin or a fragment thereof conjugated to an immunogenic protein or fragment thereof derived from a pathogenic virus, wherein exon 4, exon 5, or both exon 4 and exon 5 are not present in the N-terminal domain of osteopontin or the fragment thereof.

13. The fusion protein according to claim 12, wherein the pathogenic virus is SARS-CoV-2.

14. The fusion protein according to claim 13, wherein the fusion protein comprises SEQ ID NO: 7 or an amino acid sequence having at least 80% sequence identity with SEQ ID NO: 7.

15. (canceled)

16. A cell engineered to express the fusion protein of claim 12.

17. A nucleic acid encoding the fusion protein of claim 12.

18. (canceled)

19. A method for potentiating an immune response to an immunizing antigen in a subject, the method comprising administering to the subject an effective amount of the vaccine adjuvant according to claim 2.

20-21. (canceled)

22. The method according to claim 19, wherein the vaccine adjuvant is co-administered with the immunizing antigen, wherein the immunizing antigen is selected from the group consisting of a protein or fragment thereof, a nucleic acid, a virus, a pseudovirus, a bacterium, or a parasite, wherein the virus, the pseudovirus, the bacterium or the parasite is inactivated or attenuated.

23-24. (canceled)

25. The method according to claim 22, wherein the immunizing antigen is a viral, bacterial, or parasitic protein or fragment thereof.

26. The method according to claim 22, wherein the N-terminal domain of osteopontin or the fragment thereof and the immunizing antigen are administered concurrently or sequentially.

27. The method according to claim 22, wherein the N-terminal domain of osteopontin or the fragment thereof is conjugated to the immunizing antigen.

28-33. (canceled)

34. A cell engineered to express the vaccine adjuvant of claim 1.

35. A vaccine comprising:
the cell according to claim 34; and
a pharmaceutically acceptable carrier.

36. The vaccine according to claim 35, wherein the cell is an autologous cell obtained from a subject.

37. A method of vaccinating a subject in need thereof, the method comprising:
obtaining autologous cells from the subject;
transducing the autologous cells with a nucleic acid encoding an N-terminal domain of osteopontin, wherein exon 4, exon 5, or both exon 4 and exon 5 are not present in the N-terminal domain of osteopontin or the fragment thereof; and
reintroducing the autologous cells into the subject.

38. The method according to claim 37, wherein the N-terminal domain of osteopontin is present as part of a fusion protein comprising the N-terminal domain of osteopontin and an immunizing antigen.
